(19) United States
(12) Patent Application Publication
(10) Pub. No.: US 2020/0066379 A1
(43) Pub. Date: Feb. 27, 2020

(54) Providing all patients & all general populations medical information worldwide at all time to all health care providers using health information technology via biometric (fingerprint, iris, facial ID), Social Security, or government identification number ID/Driver's License with a PIN to be used globally primarily by hospitals, medical offices/clinics, paramedics, fire departments, law enforcement and electronic health systems to access, organize, maintain storage & to share

(71) Applicant: Myrlene Chapman, Irvine, CA (US)

(72) Inventor: Myrlene Chapman, Irvine, CA (US)

(21) Appl. No.: 16/257,065

(22) Filed: Jan. 24, 2019

Related U.S. Application Data
(60) Provisional application No. 62/621,275, filed on Jan. 24, 2018.

(51) Int. Cl. G16H 10/60; A61B 5/117; G06F 21/32

(52) U.S. Cl. CPC G16H 10/60; H04L 63/0861; G06F 21/32; A61B 5/117

(57) ABSTRACT

The application connects patient data in real time for entry and retrieval for three user profiles; patient, provider, and facility. Permissions to access and modify data are based on roles that are read/write, or read-only stored in a database or collection of databases such as a cloud-based service. Typical data retrieval will be secure authentication to access a database and return critical or non-critical information based on the defined user role.

### TITLE OF INVENTION

Providing all patients & all general populations medical information worldwide at all time to all health care providers using health information technology via biometric (fingerprint, iris, facial ID), Social Security, or government issued identification number ID/Driver's License, which a Personal Identification Number (PIN) to be used globally by healthcare professionals.

### BACKGROUND OF THE INVENTION

Here, describing some references that are related to the reasoning of the invention, which relates to some specific problems involved the state of technology: Last year, providers at hospitals and health systems across the country faced major care challenges when dealing with the aftermath of natural disasters including Hurricane Harvey, Hurricane Irma, and the northern California wildfires. The latter charred more than 200,000 acres of land across 10 counties and was declared a public health emergency by HHS Acting Secretary. Natural disasters of this magnitude can have a significant impact on patient access to care, health data access, and clinician workflows. Patients fleeing natural disasters often seek care at medical facilities they have not visited previously, which requires providers to find ways to access necessary patient health information for well-informed clinical decision-making. In addition, multiple patients medical care was delayed because of medical professionals not having medical data access during the individual business trips &/or personal trips overseas, which results to lifetime disabilities &/or death.

### SUMMARY OF THE INVENTION

Here, this application data of the electronic health records will point the advantages of the invention and how it solves previously existing problems (Example: described above in the background), which will provide all patients (infants to elderly) medical information worldwide at all times to all health care providers using health info technology via biometric (fingerprint, iris, facial ID), Social Security, or government issued identification number ID/Driver's License or company ID with a PIN. The data access will be used globally primarily by hospitals, medical offices/clinics, paramedics, fire departments, and law enforcement, and electronic health systems to access, organize, maintain storage and the ability to share in/outpatients all medical records with other medical facilities and medical providers. All patients' electronic health records information listed below will maintain confidential to follow HIPAA rules and regulations. However, if patient is unconscious without family member(s) emergency staff can obtain access with special emergency code. Therefore, when patient is conscious and non-emergency matters the inventor's company and/or associate vendors can provide a medical advocate staff upon patient's request to medical visits including emergencies.

The purpose of the invention is the access of the electronic health records information that will contain all patients hospitals/medical offices/clinics medical/surgical histories, medical and non-medical allergies: including laboratory, chemotherapy, radiation, pathology, blood type, X-rays, imaging, all physicians progress notes, nurses/other medical staff notes, physicians/PA's/NP's orders, HIPAA, advance directive, power of attorney, conservative, allied health, all medical devices (ex: pacemaker/defibrillator information, etc.), all medical departments/divisions, convulsion/strokes history, clinical research study: medical devices & study drugs, all medications history & reasons, vitamins, all medications over the counter (otc), care plans, mental health: medications/history/hospitalizations/current status, all records from outside facilities, inpatients discharge summary, outside procedures, hospitalizations, emergency department visit documentations/tests, acute rehab unit, nursing skilled facilities, family medical history, and guide to maintain all nutritional and healthcare.

| Beneficiaries | Benefits | Promoters |
|---|---|---|
| Emergency | Expedited Diagnoses | insurance Companies |
| Response Teams | | |
| Healthcare providers | Focused/Informed | State Department |
| | treatment | |
| Heath Insurance | Cost Savings | |
| General Population/ | Expedited Authorizations | |
| Patient | | |
| CDC | Early Detection | |
| University and | Timely Treatment/ | |
| Research Trials | Tractability | |
| | Avoids Trial & Error | |

What Specific Problem Does IT Solve? MONEY

| Patient | Insurance | Medical Field | Government |
|---|---|---|---|
| Expedited | Cost Savings | Fraud | CDC |
| Diagnosis | | | |
| Focused | Focused | Immediate access | Fraud |
| Treatment | Treatment | to medical history | |
| Reduce trial and | Reduce trial and | Cost savings | Opiate Crisis |
| error | error | Reduced tests | Addition |
| | | Reduced stay | Homelessness |
| | | time | Crime |
| | | Reduced ER time | |
| | | Litigations | |
| | | Complications | |
| | | Misdiagnosis | |
| Early Detection | Early Detection | | Tractability |
| Cost savings | Fraud Reduction | | |
| (time, $$, | | | |
| Stress) | | | |
| Expedited | Expedited | | |
| Authorization | Authorization | | |
| | Less time, | | |
| | less tests, | | |
| | reduced | | |
| | complications | | |
| | Reduced | | |
| | Appeals. | | |
| | Litigation | | |
| | and opiates. | | |
| Mortality | | | |
| Mitigates | | | |
| Complications | | | |

Who Would Have Access?

| General Population | Patient | Insurance Co. | Medical Field | Government |
|---|---|---|---|---|
| Password Security | Finger print | Master | Master | Master |
| Password | Retina | Subscriber | subscription | subscription (fob) |
| Verification | Facial | (fob) | (fob) | Staff fingerprint |
| Text | Recognition | Finger print | Finger print | |
| Personal | | | Patient finder | |
| Question | | | print | |
| History | | | Facial ID | |
| Question | | | | |
| Notification | Notification | | | |
| when history | when history | | | |
| is accessed | is accessed | | | |

Time savings

| General | | | Insurance | Govern- |
|---|---|---|---|---|
| Population | Patient | Medical Field | Company | ment |
| No | No Paperwork | Reduced | ADMIN | ADMIN |
| paperwork | immediate | Admin | Clean | |
| (sustainability- | informed | time | information | |
| go green) | medical | Authorization | flow | |
| | attention | reviews | | |
| | Unnecessary | Treatment | | |
| | appointments | delays | | |
| | Reduced | ER time | | |
| | Hospital | Clean | | |
| | Stay, missed | information | | |
| | work, etc. | flow | | |

Types of Mistakes Avoided

| General | | | Insurance | Govern- |
|---|---|---|---|---|
| Population | Patient | Medical Field | Company | ment |
| X | Miss | Miss Identification | X | X |
| | Diagnosis, etc. | Patient | | |
| | Wrong | Medication | | |
| | Treatment | Blood Tests | | |
| | Drug | | | |
| | Interactions | | | |
| | Allergy | | | |
| | Delayed | | | |
| | diagnosis | | | |
| | Delayed | | | |
| | Treatment | | | |
| | Infection | | | |

General Population

| Access | Visibility | Categories |
|---|---|---|
| Passport | Full Access | Blood Type |
| Verification Code | | Immunization |
| Personal question | | Travel |
| History Question | | Medical History |
| Alert when history is | | Medical Records |
| accessed | | |
| | | Surgical History |
| | | Hospitalizations |
| | | Family History |
| | | Medication |
| | | Allergy |
| | | Exposures |
| | | Disease |
| | | Airborne |
| | | food borne |
| | | Name |
| | | Age |
| | | DOB |
| | | Gender |

Patient/Medical

| Access | Visibility | Categories |
|---|---|---|
| Finger Print (Biometric) | Full Access | Blood Type |
| Retina | | Immunization |
| Facial Recognition | | Travel |
| | | Medical History |
| | | Medical Records |
| | | Surgical History |
| | | Hospitalizations |
| | | Family History |
| | | Medication |
| | | Allergy |
| | | Exposures |
| | | Disease |
| | | Airborne |
| | | food borne |
| | | Name |
| | | Age |
| | | DOB |
| | | Gender |

### DESCRIPTION OF THE INVENTION

The primary access point to patient data is an application for first responders, EMT professionals, Physicians, Physician Assistant, Nurse Practitioner, and Emergency or Urgent care providers to have real time access to patient records from anywhere in the world via internet protocols. The application will consist of a secure authentication path that will retrieve the patient record and populate both readable and writeable forms. The application will run from a mobile device, laptop, tablet, and desktop that has Internet access &/or a telecommunications company (mobile devices, etc.). Data editing may also occur from a web browser on a mobile or non-mobile device. A defined user role will be administered to prevent in-accurate information from being populated that may cause life-threatening situations in critical situations.

### Authentication:

An application user will log in a patient with information that is unique, with biometric (fingerprint, iris, facial ID), Social Security, or government issued identification number such as a driver's license number. Additional login options will be available to these unique identifiers. Biometric allows medical staff to retrieve data from incapacitated or unresponsive patients.

### Permissions/Roles:

Based on what type of user is requesting the data, they may have limited access to read only or full access to modify data. All changes made will occur in real time and populate globally. This information could also include onsite care during an event before the patient is transported to a facility for care. An example of this would be the administration of medication, transfusion or utilization of a medical device to stabilize a patient until such time as they arrive at a care facility.

### Database:

Databases of patient data will be hosted in web-accessible data warehouses that do not require human review of access, and thus enable real time retrieval in emergency or time critical situations. The database will contain patient records and other data related to healthcare and quality of life information.

### Use Cases:

1. EMT responds to an incident with a mobile device with the application running. The patient's fingerprint or iris or facial ID is scanned with a calibrated biometric scanner. The EMT secondary verifies the image on file or communicates with the patient if possible, to begin administering treatment. The application will return information about medication, medical devices or recent treatments that may be pertinent to the situation.
2. Provider or facility users will be considered the highest-level users that currently have access to create, read, or modify patient medical records. Data is “live” in that it is immediately available to all other facilities, providers and even patients in real time. This may include insurance companies as well.
3. Patients are a third user type. As a user they will be able to read most medical information, but they will also have the ability to enter data related to insurance, emergency contacts, end of life, long term care, and a will or trust Users will not be able to modify details about information that may cause conflicts in care. An example would be a patient modifying their blood type, allergies or current medications.
4. The primary access can be leverage with fingerprint, iris and facial identification for medical care, reducing opiate crisis and mortality in order to provide patients immediate medical care. In addition, patients and the general populations reviewing their medical records is not only to prevent delay of medical care, but also to the safety and smart process for the patient and their rights. Therefore, this data access allows the patient to update any information that may be vital to their care or accurately discussed information with their medical provider about prescriptions or test results that are missing or incorrect
5. Historically, medical records were kept and maintained by the primary care provider. However, in the twenty first century, a trend has emerged, which technology has advanced in order to enhance the care of patients and the patients willing to take responsibility for the storage and maintenance of their own medical records.

1. This software application data connection initiated (eg: WiFi, Mobile Data, Tablet computer, Personal digital assistants [PDA's], personal organizers or the likes).

2. This software application launched according to claim 1, wherein patients with an identification verification (fingerprint/biometric scan or unique ID) will have access to see portions of their records as well as the ability to modify limited fields, related to insurance information, new address and phone number, emergency contacts, medical directives/power of attorney, prescriptions or over the counter [otc] updates, allergies, notes to their healthcare providers.

3. This software application User Logs in according to claim 1, wherein first responders/EMT/law enforcement with a PIN code will have access to immediate patient data that would be relevant to their urgent/emergency care, which the data would be read only and will alert on recent prescriptions, allergies, surgeries, healthcare providers, emergency contacts and medical devices information.

4-12. (canceled)

4. This software application user logs in according to claim 2, wherein healthcare providers will have limited write access, but unrestricted read access to patient records, which some maybe granted full access as proxy for a physician, and pharmaceutical providers will be able to signal when a prescription has been dispensed.

5. This software application user logs in according to claim 2, wherein medical insurance companies will have limited access to patient records with the ability to describe coverage, limits, benefits billing, and emergency authorizations.

6. This software application user logs in according to claim 2, wherein institution/research (CDC, FDA, medical device/pharmaceutical companies, universities and other known governmental/nongovernmental agencies), whereas patients are able to volunteer data in addition to other non-identifiable data to be used for statistical analysis of events, outbreaks, and health trends.

7. This software application user logs in according to claim 2, wherein the patient request information goes to access layer routed to the data warehouse/icloud, whereas the data is placed in the appropriate application form/template, read-only, modifiable fields, the data is edited and changes are written to the application with the date, time and owner's identity.

8. This software application user logs in according to claim 4, wherein the healthcare provider request information goes to access layer routed to the data warehouse/icloud, whereas the data is placed in the appropriate application form/template read-only, modifiable fields, the data is edited and changes are written to the application with the date, time and owner's identity.
